(19) United States
(12) Patent Application Publication
(10) Pub. No.: US 2008/0057022 A1
(43) Pub. Date: Mar. 6, 2008

(54) Ophthalmic Pharmaceutical Compositions and Uses Thereof

(76) Inventor: Erning Xia, Penfield, NY (US)

Correspondence Address: Bausch & Lomb Incorporated, One Bausch & Lomb Place, Rochester, NY, 14604-2701, US

(21) Appl. No.: 11/842,394

(22) Filed: Aug. 21, 2007

Related U.S. Application Data
(60) Provisional application No. 60/841,036, filed on Aug. 30, 2006.

(51) Int. Cl. A61K 31/74; A61P 27/02

(52) U.S. Cl. 424/78.04

(57) ABSTRACT

A composition for providing relief to a discomfort of an eye comprises a pharmaceutically acceptable carrier and a material that allows the composition to remain in an ocular environment for an extended period of time. The composition can further comprise an active ingredient selected from the group consisting of anti-allergic agents, anti-inflammatory agents, anti-infective agents, and combinations thereof.

### CROSS-REFERENCE

This application claims the benefit of Provisional Patent Application No. 60/841,036 filed Aug. 30, 2006, which is incorporated by reference herein.

### BACKGROUND OF THE INVENTION

The present invention relates to ophthalmic pharmaceutical compositions and their use to provide relief to eye conditions.

Many environmental factors can negatively affect the health of the eye. For example, allergens, irritants (such as chemicals), viruses, and bacteria can result in redness, irritation, or inflammation of the conjunctiva. Diseases from other parts of the body also can affect the eye. Dry eye (also known as keratoconjunctivitis sicca (“KCS”)), a disorder manifested as insufficient tear film for lubrication of the ocular surface, can be the result of environmental factors or diseases. Dry eye affects millions of people each year. There has been increasing evidence that inflammation may be an important factor in the pathogenesis of KCS. For example, inflammation of the lacrimal and meibomian glands can curb tear production. In addition, elevated levels of pro-inflammatory mediators, including IL-1, have been detected in the conjunctival tissues of patients afflicted with systemic autoimmune diseases, such as Sjogren's syndrome. Sjogren's syndrome is a chronic disorder in which white blood cells attack the moisture-producing glands, such as lacrimal and salivary glands. Dry eye may afflict individuals with differing severity. In mild cases, a patient may experience burning, a feeling of dryness, and other symptoms of ocular discomfort. In severe cases, vision may be substantially impaired. Although dry eye may have a variety of unrelated pathogenic causes, they all share as a common effect the breakdown of the ocular tear film, with dehydration of and subsequent damage to the exposed outer ocular surfaces.

These pathological conditions produce significant discomfort in the eye, such as itching or burning sensation, which can benefit from relief by palliative agents. Many such agents have been provided with varying success. The relief that they provide are often short-lived. Therefore, there is a continued need to provide improved ophthalmic compositions for the relief of ocular discomfort. In addition, it is very desirable to provide such compositions for an extended relief of ocular discomfort.

### SUMMARY OF THE INVENTION

In general, the present invention provides ophthalmic pharmaceutical compositions for relieving discomfort in an eye.

In one aspect, such discomfort results from an ocular condition, such as allergy, infection, inflammation, or dry eye syndrome.

In another aspect, such discomfort may result from a treatment of another condition of the eye, such as an eye surgery (e.g., glaucoma surgery, cataract surgery, or surgery to treat a back-of-the-eye condition).

In still another aspect, an ophthalmic pharmaceutical composition of the present invention comprises a material that allows the composition to remain on an ocular surface for an extended period of time.

In still another aspect, an ophthalmic pharmaceutical composition of the present invention comprises a demulcent.

In yet another aspect, an ophthalmic pharmaceutical composition of the present invention is devoid of preservatives that can produce discomfort in the eye.

In a further aspect, an ophthalmic pharmaceutical composition of the present invention is devoid of preservatives.

In yet another aspect, the present invention provides a method for relieving discomfort in an eye, the method comprising administering into an affected eye a composition that comprises a material that allows the composition to remain on an ocular surface for an extended period of time and that is devoid of preservatives that can produce other discomfort in said eye.

Other features and advantages of the present invention will become apparent from the following detailed description and claims.

### DETAILED DESCRIPTION

In one aspect, compositions of the present invention comprise ingredients and are prepared according methods as shown in the following description.

### EXAMPLE 1

### Bausch & Lomb Relief Extra Strength Preservative Free Product BL-700-DDE03

### Description

Formulation and Compounding Procedure for Advanced Eye Relief Extra Strength Preservative Free (BL-700-DDE03)

### Materials and Equipment

Pre-Filters [symbol] Sartorius 20 μm Sartopure PP2

Sterilizing Filters [symbol] Sartorius 0.2 μm Sartopore 2

Polishing Filter [symbol] Millipore CR75

### Formulation

| Specification | Ingredient, Component, or | | |
|---|---|---|---|
| Number | Intermediate Product | mg/gm | % w/w |
| ER-5272 | Carbopol 980NF | 0.620 | 0.0620 |
| RM-1972 | Glycerin | 2.000 | 0.2000 |
| ER-5275 | Sorbitol | 36.660 | 3.6660 |
| RM-1816 | Sodium Hydroxide NF | 0.157 | 0.0157 |
| | Pellets | | |
| RM-1806 | Purified Water | q.s. to 1 gm | q.s. to 100% |

### Equipment Preparation

Using a validated cycle(s), perform a steam-in-place sterilization on the main batch tank, 20 μm pre-filter, 0.2 μm sterilizing filter and aseptic transfer lines. The main batch tank should have the capacity to accommodate the entire batch weight.

### Compounding Procedure

Phase 1
1) Charge a clean and approved tank with purified water to 40-50% of the total batch weight. Adjust the temperature to 15-25° C.
2) When initial Phase 1 charge is complete, as verified by the flow meter, turn on the agitator.
3) Slowly add and disperse the following ingredients in the order listed. A mechanical dispersion device such as an In-line SLIM or Eductor or equivalent may be used to improve the addition rate and dispersion of the material. After the addition is complete, begin recirculation of batch. [symbol] Carbopol 980 NF
4) Mix the dispersion for NLT 30 minutes.
5) Visually inspect the dispersion to ensure that there are no clumps or agglomerates.
6) Secure the mix tank manway and close all tank headspace bleed valves.
7) Initiate product heat up.
8) When solution temperature reaches 115° C., open the tank bleed valves and steam injection valve to begin the steam injection of the head space.
9) Maintain steam injection pressure in the tank headspace at 15 to 27 psig during the autoclave phase.
10) Continue ramp up sequence into the product sterilization phase. Sterilization will be 121° C.-124° C. for 30-40 minutes.
Note: Maintain sterilization temperature within the parameters specified in (Phase 1, step 10). Total sterilization time must not exceed 40 minutes.
11) Upon completion of the sterilization cycle, initiate batch cooling, close tank bleeds, discontinue steam injection.
12) Cool the product to between 20° C. and 30° C. and hold while maintaining agitation.
Note: Tank must be maintained under positive pressure with sterile air during cool down, storage and filling activities.
13) Filter the Phase 1 Carbomer material into the main mix tank through the sterile transfer line and the 20 μm Sartorius 5592520P1 PP2 clarifying filter.

### Phase 2

1) Charge a clean and approved tank with purified water to 40-50% of the total batch weight. Adjust the temperature to 40° C.±3° C.

2) Initiate agitation then add the following raw materials to the batch as listed, allowing each to dissolve completely, as noted visually, before adding the next: [symbol] [symbol]
3) Allow solution to mix thoroughly for not less than 20 minutes
4) Adjust the temperature of the solution to 25° C±3° C. Aseptically transfer the mixed solution into the main mix tank through the sterile transfer line and the 0.22 μm Sartorius PES 5442507H1 sterile addition filter.

### Phase 3

Note: Take care to reduce splashing while handling caustic materials. Wear proper personal protective equipment.
1) Charge an appropriately sized 316 stainless steel vessel with an appropriate quantity of Purified Water between 3%-5% of the total batch weight to bring the batch quantity to final batch weight. Adjust Purified Water temperature to 20-25° C.
2) Initiate agitation then add:
[symbol] Sodium Hydroxide Pellets
3) Allow the Sodium Hydroxide to dissolve completely.
4) Aseptically transfer the Sodium Hydroxide solution into the main mix tank through the sterile transfer line and the 0.22 μm Sartorius PES 5442507H1 sterile addition filter.
5) Allow solution to mix thoroughly for not less than 30 minutes. Final product batch temperature must be <30° C.
6) Aseptically transfer the final solution through a sterile transfer line and a sterile 75 μm Millipore CR75 polypropylene polishing filter. During the transfer and post polishing filter, aseptically withdraw a minimum 300 ml sample and submit to Quality Control for in-process testing.

### Sampling and In-Process Testing

1) A minimum 300 ml sample (properly identified) must be aseptically drawn and submitted to Quality Control for testing.

| | | Tentative Acceptance |
|---|---|---|
| Test | Test Methodology | Criteria |
| pH @ 25° C. | TP-7801 | 6.6-7.4 |
| Color | TP-8090 | 0-30 APHA |
| Clarity | TP-8158 | Clear |
| Specific Gravity | TP-7875 | Approx. 1.014 |
| Osmolality | TP-7803 | 220-260 mOsm/kg |

2) If any test is questionable or any adjustments to the solution are made, a second sample shall be submitted for testing (per bullet 1).

### Product Filling

1) The temperature of the product to be filled should be between 20° and 30° C.

2) Aseptically fill appropriate primary packaging configuration under Class 100 conditions.

### Product Label Claim

| Ingredients | % w/w |
|---|---|
| Glycerin | 0.20 |

### Contract Manufacturer Responsibilities

If any change in raw materials or processing methods is under consideration by the manufacturer, the manufacturer must notify Bausch & Lomb prior to the implementation of the change and no change may be made without written approval by Divisional Quality.

### Control of Nonconforming Product and Corrective Action

Refer to BL-POL-1301, “Handling Deviations and Variances,” for specific information on how to handle product that does not meet specification.

### EXAMPLE 2

### Bausch & Lomb Advanced Eye Relief Long Lasting (Preservative Free) Product BL-700-DDE09

### Description

Advanced Eye Relief Long Lasting product (BL-700-DDE09) is a sterile, buffered, preservative free, hypotonic solution intended for use as an artificial tear and lubricant for providing soothing therapy to dry irritated eyes. The solution is a non-blurring, low viscosity liquid that contains propylene glycol and glycerin as demulcents which lubricate and soothe the irritated corneal epithelium. The solution also contains alginate, a hydrocolloid, which serves to interact with the mucin layer in the tear film and holds moisture for a long time. This helps to keep the tear film intact and provides long term relief to the dry eyes.

The product is manufactured as a single phase process and batch transferred to a sterile hemisphere through 0.22 μm sterile elements. The product is packaged in a single dose unit configuration via a form-fill-seal process.

### Materials and Equipment

Sterilizing filter: Millipore CVGL71TP3 (0.22 micron) hydrophilic filter. Formulation: (No. BL-700-DDE09)

| Specification | Ingredient, Component, or | | |
|---|---|---|---|
| Number | Intermediate Product | mg/gm | % w/w |
| RM-3647 | Protanal LF200M Alginate, | 2.50 | 0.25 |
| | medium viscosity | | |
| RM-1972 | Glycerin | 6.00 | 0.600 |
| RM-3001 | Propylene Glycol | 6.00 | 0.600 |
| RM-1804 | Sodium Borate | 0.14 | 0.014 |
| RM-1812 | Boric Acid | 5.00 | 0.500 |
| RM-3240 | HAP (30%) (hydroxyalkyl | 0.50 | 0.050 |
| | phosphonates) | | |
| RM-1806 | Purified Water, USP | Q.S. to | Q.S. to |
| | | 1000.0 mg | 100% w/w |

### Compounding Procedure

1) Add purified water (40°-45° C.) into an appropriate size 316 stainless steel jacketed mixing vessel, equipped with agitation. The amount should be sufficient to cover the mixing blades but not more than the batch weight requirement. Record the amount of water used.
2) Initiate agitation. Note: Optimally, use a minimal purified water quantity for achieving maximum agitation. Set agitator at a maximum setting that does not cause splashing.
3) Slowly add the following raw materials to the batch in the order listed, allowing each to dissolve or disperse before adding the next ingredient. [symbol] RM-3240, HAP 30%
[symbol] RM-1972, Glycerin
[symbol] RM-3001, Propylene Glycol
4) Mix the solution for not less than 30 minutes. Maintain the temperature not more than 45° C.
5) Dry blend the following ingredients together for not less than 15 minutes. Blend should not contain clumps.
[symbol] RM-1812, Boric Acid
[symbol] RM-1804, Sodium Borate
[symbol] RM-3647, Protanal LF200M Alginate, medium viscosity
6) Add the blend and continue mixing the solution.
7) Bring the batch to final weight with purified water. Water temperature should not exceed 45° C. Record the amount of water used.
8) Continue to mix the solution for not less than 60 minutes. Maintain the temperature between 40° C. and 45° C.
9) Reduce the temperature to between 20° C. and 25° C. Continue to mix the solution for not less than 20 minutes.
10) Sample the batch per the Sampling and In-Process Testing section and submit to Quality Control.
11) Aseptically transfer the solution to the sterile hemisphere through the sterile 0.22 micron filter. Refer to MAP-6013, General Procedure for Solutions Sterilized by Filtration, for process and documentation requirements.

### Sampling and In-Process Testing

Test the sample for the following:

| Test | Test Methodology | Acceptance Criteria |
|---|---|---|
| pH @ 25° C. | TP-7801 | 6.7-7.1 |
| Osmolality | TP-7803 | 200-260 mOsm/kg |
| Color | TP-8090 | 0-50 APHA |
| Clarity | TP-8158 | clear |

If any test is questionable, or if any adjustments to the solution are made, a second sample shall be submitted for testing (per section Sampling and In-Process Testing).

### Product Label Claim

| Ingredients | % w/w |
|---|---|
| Glycerin | 0.6 |
| Propylene Glycol | 0.6 |

### Filing

After release by Quality Control, aseptically fill product into single dose unit vials under class 100 conditions. Fill single dose unit vial to no less than 0.80 ml.

### Additional Requirements

If any change in raw materials or processing methods is under consideration by the manufacturer, the manufacturer must notify Bausch & Lomb prior to the implementation of the change and no change may be made without written approval by Divisional Quality.

### Contract Manufacturer Responsibilities

If any change in raw materials or processing methods is under consideration by the manufacturer, the manufacturer must notify Bausch & Lomb prior to the implementation of the change and no change may be made without written approval by Divisional Quality.

### Control of Nonconforming Product and Corrective Action

Refer to BL-POL-1301, “Handling Deviations and Variances,” for specific information on how to handle product that does not meet specification. References

BL-POL-901, Master Records and Production History Records. 7026-FDP-163 (rev 0), Formulation Development Procedure for Drop Dry Eye

In another aspect, a material that allows the composition to remain on an ocular surface for an extended period of time after the composition has been administered to said ocular surface can be a demulcent. As used herein, the phrase “an extended period of time” means a period of time of at least 1 hour. A demulcent can be a material that is water soluble, sparingly soluble, or substantially soluble in water. A demulcent that is sparingly soluble or substantially insoluble in water and still absorb water. In one embodiment, such a material is selected from the group consisting of lightly cross-linked, carboxyl-containing polymers that are substantially insoluble or only sparingly soluble in water. In another embodiment, the material is selected from the group consisting of cellulose derivatives (such as sodium carboxymethyl cellulose, hydroxyethyl cellulose, hydroxypropylmethyl cellulose, or methyl cellulose), dextran 70, gelatin, polyethylene glycol, propylene glycol, polyvinyl alcohol, polyvinylpyrrolidone, polyacrylic acid, polymethacrylic acid, combinations thereof, and mixtures thereof. A preferred material comprises a Carbopol polymer, such as Carbopol 980 or Carbopol 976. Other suitable materials include, but are not limited to, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to, or instead of, acrylic acid.

Such polymers may be crosslinked by a polyfunctional crosslinking agent, preferably a difunctional crosslinking agent. The amount of crosslinking should be sufficient to form polymers that are substantially insoluble or sparingly soluble in water. Typically the polymers are only lightly crosslinked. Preferably, the crosslinking agent is used in an amount of from about 0.01% to about 5%, preferably from about 0.1% to about 2%, and more preferably from about 0.2% to about 1%, based on the total weight of monomers present. Included among such crosslinking agents are non-polyalkenyl polyether difunctional crosslinking monomers such as divinyl glycol; 2,3-dihydroxyhexa-1,5-diene; 2,5-dimethyl-1,5-hexadiene; divinylbenzene; N,N-diallylacrylamide; N,N-diallymethacrylamide and the like. Also included are polyalkenyl polyether crosslinking agents containing two or more alkenyl ether groupings per molecule, preferably alkenyl ether groupings containing terminal H₂C═C< groups, prepared by etherifying a polyhydric alcohol containing at least four carbon atoms and at least three hydroxyl groups with an alkenyl halide such as allyl bromide or the like; e.g., polyallyl sucrose, polyallyl pentaerythritol, or the like; see, e.g., Brown U.S. Pat. No. 2,798,053, the entire contents of which are incorporated herein by reference. Diolefinic non-hydrophilic macromeric crosslinking agents having molecular weights of from about 400 to about 8,000, such as insoluble di- and polyacrylates and methacrylates of diols and polyols, diisocyanate-hydroxyalkyl acrylate or methacrylate reaction products of isocyanate terminated prepolymers derived from polyester diols, polyether diols or polysiloxane diols with hydroxyalkylmethacrylates, and the like, can also be used as the crosslinking agents; see, e.g., Mueller et al. U.S. Pat. No. 4,192,827 and 4,136,250, the entire contents of each patent being incorporated herein by reference.

Such a polymer is present in a composition of the instant invention in an amount from about 0.01 to about 5% (by weight). Alternatively, such a polymer is present in an amount from about 0.01 to about 2%, or from about 0.01 to about 1%, or from about 0.01 to about 0.5%, or from about 0.01 to about 0.2%, or from about 0.01 to about 0.1% (by weight).

In one embodiment, a composition of the present invention is in a form of a suspension or dispersion. In another embodiment, the suspension or dispersion is based on an aqueous solution. For example, a composition of the present invention can comprise sterile saline solution. In still another embodiment, the suspension or dispersion is an oil-based formulation. For example, the formulation can include an oil selected from the group consisting of vegetable oil, peanut oil, olive oil, coconut oil, sesame oil, cottonseed oil, corn oil, sunflower oil, fish-liver oil, arachis oil, liquid paraffin, and mixtures thereof.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In addition, a composition of the present invention can include additives such as buffers, diluents, carriers, adjuvants, or excipients. Any pharmacologically acceptable buffer suitable for application to the eye may be used. Other agents may be employed in the composition for a variety of purposes. For example, buffering agents, co-solvents, humectants, emollients, stabilizers, or antioxidants may be employed. Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 2 and about 11. As such the buffering agent may be as much as about 5% on a weight to weight basis of the total composition. Electrolytes such as, but not limited to, sodium chloride and potassium chloride may also be included in the formulation.

In one aspect, the pH of the composition is in the range from about 4.5 to about 11. Alternatively, the pH of the composition is in the range from about 6 to about 9, or from about 6.5 to about 8. In another aspect, the composition comprises a buffer having a pH in one of said pH ranges.

In another aspect, the composition has a pH of about 7. Alternatively, the composition has a pH in a range from about 7 to about 7.5.

In still another aspect, the composition has a pH of about 7.4.

In a further aspect, a composition of the present invention formulated for the treatment of dry eye-type diseases and disorders may also comprise carriers designed to provide immediate, short-term relief of dry eye-type conditions. Such carriers can be formulated as a phospholipid carrier or an artificial tears carrier, or mixtures of both. A phospholipid carrier comprises one or more phospholipids that lubricate, wet, approximate the consistency of endogenous tears, aid in natural tear build-up, or otherwise provide temporary relief of dry eye symptoms and conditions upon ocular administration. Non-limiting examples of phospholipid carrier formulations include those disclosed in U.S. Pat. Nos. 4,804,539; 4,883,658; 4,914,088; 5,075,104; 5,278,151; 5,294,607; 5,371,108; 5,578,586; the foregoing patents are incorporated herein by reference to the extent they disclose phospholipid compositions useful as phospholipid carriers of the present invention.

In yet another aspect, a composition also can comprise a viscosity-modifying compound designed to lubricate, wet, approximate the consistency of endogenous tears, aid in natural tear build-up, or otherwise provide temporary relief of dry eye symptoms and conditions upon ocular administration the eye. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cps”), or from about 1 to about 200 cps, or from about 1 to about 100 cps. In one embodiment, a viscosity-modifying compound is water soluble.

In still another aspect, a method for preparing a composition of the present invention comprises combining at least a material that allows the composition to remain in an ocular environment (such as on an ocular surface) for an extended period of time with a pharmaceutically acceptable carrier. In one embodiment, such a carrier can be a sterile saline solution or a physiologically acceptable buffer.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5× PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C, and pH in the range of about 7-8.2; TAPS (1{(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having a slight acidic pH, such as from about 6 to about 6.8. In such embodiments, the buffer capacity of the composition desirably allows the composition to come rapidly to a physiological pH after being administered to into the patient. Alternatively, a composition of the present invention has a pH from about 6.5 to about 7.5.

In still another aspect, a method for providing relief to a discomfort of the eye, comprises: (a) providing a composition comprising a material that allows the composition to remain in an ocular environment (such as on an ocular surface) for an extended period of time; and (b) administering to said eye an amount of the composition at a frequency sufficient to provide relief to said discomfort of said eye.

In another aspect, a composition of the present invention is administered topically under an eyelid or on the ocular surface of the subject. In still another aspect, a composition of the present invention is injected into the conjunctival tissue of the subject.

In yet another aspect, a composition of the present invention is administered topically once daily, several times per day, once every other day, or once a week, as necessary to provide relief to a discomfort of the eye.

In a further aspect, a composition of the present invention can comprise an active ingredient selected from the group consisting of anti-allergic agents, anti-inflammatory agents, anti-infective agents. Such an active ingredient can be present in an amount from about 0.001 to about 2% (by weight) (or from about 0.001 to about 1%, or from about 0.01 to about 0.5%, or from about 0.01 to about 0.2%, or from about 0.01 to about 0.1% by weight).

Non-limiting examples of anti-allergic agents include antihistamines, mast-cell stabilizers, and combinations thereof.

Non-limiting examples of anti-infectives include antibacterial agents, antfungal agents, antiviral agents, antiprotozoal agents, and combinations thereof.

Non-limiting examples of anti-inflammatory agents include non-steroidal anti-inflammatory drugs (“NSAIDs”), glucocorticoids, antagonists to or inhibitors of proinflammatory cytokines, and combinations thereof.

Examples of these active ingredients may be found in “Goodman & Gilman's The Pharmacological Basis of Therapeutics,” L. L. Brunton et al. (Eds), 11^{th} ed., McGraw-Hill Publ., New York, N.Y. (2006).

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising a material that is capable of allowing the composition to remain in an ocular environment for an extended period of time after said composition has been administered thereto, wherein said composition has a osmolality of about 200-260 mOsm/kg, and a pH of about 6.5-7.5.

2. The composition of claim 1, wherein said material comprises a carboxyl-containing polymer.

3. The composition of claim 2, wherein said carboxyl-containing polymer comprises from about 0.01 to about 5% by weight of the composition.

4. The composition of claim 3, wherein said polymer comprises polyacrylic acid.

5. The composition of claim 4, wherein the composition is devoid of preservatives that produce other discomfort to the eye.

6. The composition of claim 1, further comprising a viscosity-modifying compound.

7. The composition of claim 6, wherein said viscosity-modifying compound is selected from the group consisting of glycerol, cellulose derivatives, and combinations thereof.

8. The composition of claim 7, wherein the composition is devoid of preservatives that produce other discomfort to the eye.

9. The composition of claim 2, further comprising a viscosity-modifying compound.

10. The composition of claim 2, further comprising an active ingredient selected from the group consisting of anti-allergic agents, anti-inflammatory agents, anti-infective agents, and combinations thereof.

11. The composition of claim 6, further comprising an active ingredient selected from the group consisting of anti-allergic agents, anti-inflammatory agents, anti-infective agents, and combinations thereof.

12. A method for providing relief to a discomfort of an eye, the method comprising administering to an environment of an affected eye a composition that comprises a material that is capable of allowing the composition to remain in an ocular environment for an extended period of time after said composition has been administered thereto, wherein said composition has a osmolality of about 200-260 mOsm/kg, and a pH of about 6.5-7.5, in an amount and at a frequency sufficient to provide said relief.

13. The method of claim 12, wherein said carboxyl-containing polymer comprises from about 0.01 to about 5% by weight of the composition.

14. The method of claim 13, wherein said polymer comprises polyacrylic acid.

15. The method of claim 12, further comprising a viscosity-modifying compound.

16. The method of claim 15, wherein said viscosity-modifying compound is selected from the group consisting of glycerol, cellulose derivatives, and combinations thereof.

17. A method for producing a composition that is capable of providing relief to a discomfort of an eye, the method comprising combining a pharmaceutically acceptable carrier with a material that is capable of allowing said composition to remain in an ocular environment for an extended period of time after said composition has been administered to said ocular environment, to produce said composition, wherein said composition has osmolality of about 200-260 mOsm/kg and pH of about 6.5-7.5.

18. The method of claim 17, wherein said carboxyl-containing polymer comprises from about 0.01 to about 5% by weight of the composition.

19. The method of claim 18, wherein said polymer comprises polyacrylic acid.

20. The method of claim 17, further comprising including a viscosity-modifying compound in said composition.

21. The method of claim 20, wherein said viscosity-modifying compound is selected from the group consisting of glycerol, cellulose derivatives, and combinations thereof
